(19) United States
(12) Patent Application Publication
(10) Pub. No.: US 2012/0141322 A1
(43) Pub. Date: Jun. 7, 2012

(54) UV SANITIZATION AND STERILIZATION APPARATUS AND METHODS OF USE

(76) Inventor: Benjamin Fogg, Holland, MI (US)

(21) Appl. No.: 13/274,924

(22) Filed: Oct. 17, 2011

Related U.S. Application Data
(60) Provisional application No. 61/393,903, filed on Oct. 17, 2010.

(51) Int. Cl. A61L 2/10; A61L 2/00

(52) U.S. Cl. 422/24; 250/492.1

(57) ABSTRACT

An ultraviolet sanitation and sterilization apparatus having one or more of the disclosed structural, functional, and/or ornamental characteristics.

### CROSS-REFERENCE TO RELATED APPLICATION(S)

This application claims the benefit of U.S. Provisional Application Ser. No. 61/393,903, filed Oct. 17, 2010, entitled “UV Sanitization and Sterilization Apparatus and Methods of Use,” which is hereby incorporated herein by reference in its entirety, including all references cited therein.

### BACKGROUND OF THE INVENTION

1. Field of the Invention

The present invention relates in general to a UV sanitization and sterilization apparatus and method and, more particularly, but not by way of limitation, to a UV sanitization and sterilization apparatus and method for effectively monitoring and operating the UV source of the apparatus at substantially maximum output levels.

2. Background Art

UV sanitization and sterilization apparatuses have been known in the art for years, and are the subject of numerous patents, including: U.S. Pat. No. 7,160,566 entitled “Food surface sanitation tunnel,” U.S. Pat. No. 6,911,177 entitled “Ultraviolet area sterilizer and method of area sterilization using ultraviolet radiation,” U.S. Pat. No. 6,464,760 entitled “Ultraviolet air purifier,” U.S. Pat. No. 6,348,151 entitled “Device for sterilizing and filtering water which flows through a sanitary device,” U.S. Pat. No. 5,894,130 entitled “ Ultraviolet sterilization unit,” and U.S. Pat. No. 4,156,652 entitled “Apparatus for sterilizing fluids with UV radiation and ozone”—all of which are hereby incorporated herein by reference in their entirety including the references cited therein.

U.S. Pat. No. 7,160,566 appears to disclose a modular, adjustable, easy to maintain, portable or fixed food sanitation tunnel, comprising an enclosing means for subjecting food to sanitizers including UV light, ozone, hydroperoxides, superoxides and hydroxyl radicals, and a method for using the system. The enclosing means includes one or more UV radiation sources and one or more target rods located within a tunnel, such as a c-shaped shell. The UV radiation sources are preferably UV light sources that emit UV light of approximately 185 to 254 nm. The target rods are approximately up to 0-30% titanium dioxide, up to 0-30% silver and up to 0-30% copper, by weight. The system may include a mister for the efficient production of hydroxyl radicals by the UV light sources. Parts of the system are easily removable for cleaning and for maintenance. Also, in an alternative embodiment, the tunnel is located on a frame, and the frame is on wheels.

U.S. Pat. No. 6,911,177 appears to disclose an ultraviolet area sterilizer (UVAS) that is mobile or stationary. The UVAS is positioned in a room, such as an operating room or intensive care unit. Motion detectors sense movement, to assure that personnel have evacuated the space to be sterilized. Subsequently, UV-C generators, such as mercury bulbs, generate UV-C from multiple locations within the room or other enclosed space. Multiple UV-C sensors scan the room, and determine the area reflecting the lowest level of UV-C back to the sensors. The device calculates the time required to obtain a bactericidal dose of UV-C reflected back to the sensors. Once an effective bactericidal dose has been reflected to all the sensors, the unit notifies the operator and shuts down.

U.S. Pat. No. 6,464,760 appears to disclose a portable air sterilization and filtration apparatus for removing contaminants from the ambient atmosphere, having a housing with an inlet opening and an outlet opening, filter media and ultraviolet light source, and a motorized fan for maintaining a flow of air through the housing from the inlet opening to the outlet opening. The invention also includes easy access to the filter medium and to the ultraviolet light sources for periodic replacement or cleaning, and integrates a safety lock feature whereby the removal of the filter or the removal of an ultraviolet light source would open the power circuit to the ultraviolet light source, preventing accidental irradiation of the user. The ultraviolet light sources also activate an indicator light viewable by the user when the ultraviolet light sources are energized. The invention employs a three filter media system to remove contaminants from the air stream generated within the device by the motorized fan, including a sponge filter, a HEPA type filter (high efficiency particulate air filter), which will remove 99.97% of the airborne particles of the size of 0.3 microns or larger, and an activated charcoal filter. The ultraviolet light source is disposed so as to irradiate the downstream side of the activated charcoal filter during operation of the unit to provide germicidal activity to the filter's downstream surface and to the air stream as the air stream emerges from the filter prior to its discharge through the outlet opening to return to the ambient. atmosphere.

U.S. Pat. No. 6,348,151 appears to disclose a device for sterilizing and filtering water which flows through a sanitary device which consists of a treatment cavity (19) located inside of housing (2,3,4) through which water can flow. The treatment cavity is subdivided into a multitude of partial cavities (51a, 51b) by a suitably formed filter device (50). The flowing water and filter device (50) are irradiated by an ultraviolet lamp (12) and the filter device is made of a single sintered body which transmits UV radiation thereby allowing the water to be filtered to remove microorganisms and then to be killed.

U.S. Pat. No. 5,894,130 appears to disclose an ultraviolet sterilization unit having a housing attached to an air heating and cooling system. The housing including two apertures into which lamp cartridges are inserted. The lamp cartridges carrying ultraviolet lamps operating in a frequency capable of sterilizing air within the system. The cartridges are configured to automatically de-energize the lamps when a lamp cartridge is removed from the housing. When the sterilization unit is a multiple lamp system, when one of the lamp cartridges is removed all lamps are de-energized. The de-energizing of the lamps occurring before a user will view the lamp.

U.S. Pat. No. 4,156,652 appears to disclose an apparatus for sterilizing fluids comprises a radiation chamber which comprises a source of ultraviolet radiation; a housing surrounding said source and including an inner casing permeable to ultraviolet radiation and bounding a channel with said source, and an outer casing surrounding said inner casing and forming a treating space therewith; a conduit for conducting a stream of gas containing molecular oxygen through said channel for exposure to said ultraviolet radiation to produce an ozone-enriched gas; a conduit for conducting a fluid through said treating space so as to become sterilized by the ultraviolet radiation; and a conduit for introducing at least a portion of said ozone-enriched gas from said channel into said treating space to become united with said fluid, whereby said introduced portion of ozone-enriched gas is again exposed to ultraviolet radiation in order to produce an increased content of ozone in said ozone-enriched gas and an additional sterilizing effect is produced in said fluid. A process for sterilizing fluid is also provided. According to this process, a fluid and a gas which contains molecular oxygen are irradiated with ultraviolet radiation and are then mixed together in order to produce an additional sterilizing effect.

While the above-identified patents and publications do appear to provide UV sanitization and/or sterilization apparatuses, their configurations remain non-desirous and/or problematic inasmuch as, among other things, none of the above-identified apparatuses appear to be configured to effectively monitor and maintain the UV source of the apparatus at substantially maximum output levels—among other things.

It is therefore an object of the present invention to provide a UV sanitization and sterilization apparatus and method, which, among other things, remedies the aforementioned detriments and/or complications associated with the use of the above-identified, conventional UV sanitation and/or sterilization apparatuses, in particular effectively monitoring and maintaining the UV source at substantially maximum output levels.

These and other objects of the present invention will become apparent in light of the present specification, claims, and drawings.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be further understood that the invention is not necessarily limited to the particular embodiments illustrated herein.

The invention will now be described with reference to the drawings wherein:

FIG. 1 of the drawings is a perspective view of a UV sanitization and sterilization apparatus, constructed in accordance with the present invention;

FIG. 2 of the drawings is a partial perspective view of a UV source;

FIG. 3 of the drawings is a partial perspective view of the UV sanitization and sterilization apparatus of FIG. 1 in an opened configuration;

FIG. 4 of the drawings is a side perspective view of the UV sanitization and sterilization apparatus of FIG. 1;

FIG. 5 of the drawings is a partial front perspective view of the UV sanitization and sterilization apparatus of FIG. 1;

FIG. 6 of the drawings is a perspective view of a hydraulic assembly of the UV sanitization and sterilization apparatus;

FIG. 7 of the drawings is a partial perspective view of an alternative embodiment of a UV sanitization and sterilization apparatus in a closed configuration;

FIG. 8 of the drawings is a partial perspective view of an alternative embodiment of a UV sanitization and sterilization apparatus in an open configuration; and

FIG. 9 of the drawings is a perspective view of a gravity fed UV sanitization and sterilization apparatus, constructed in accordance with the present invention.

### DETAILED DESCRIPTION OF THE INVENTION

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings with like reference characters.

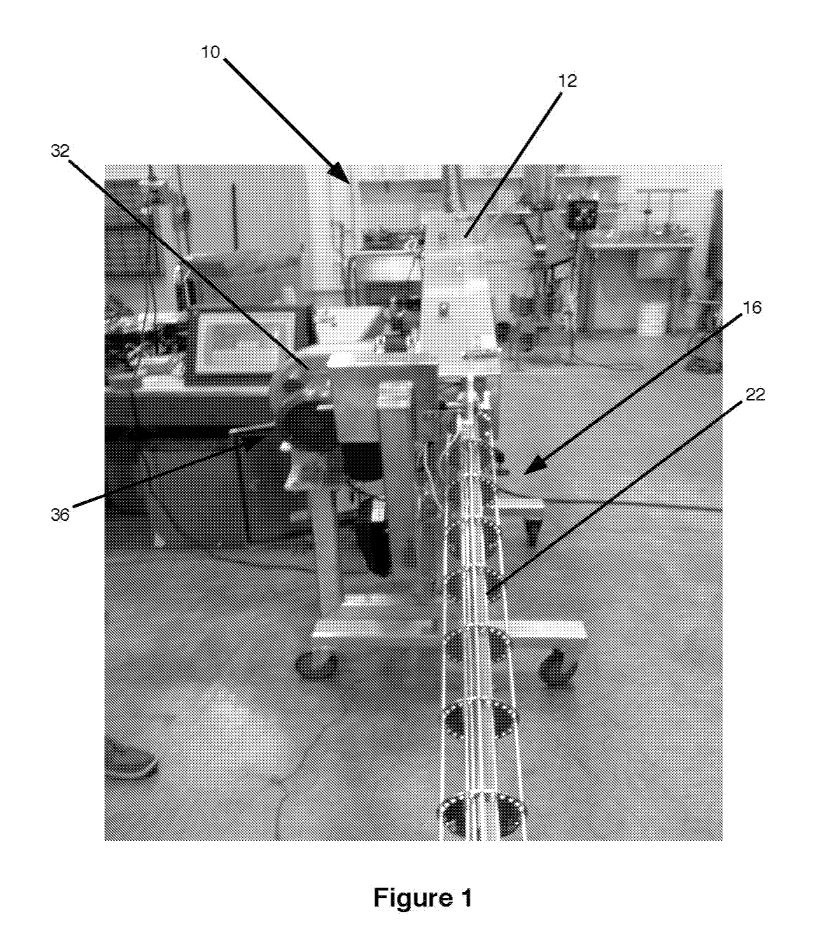

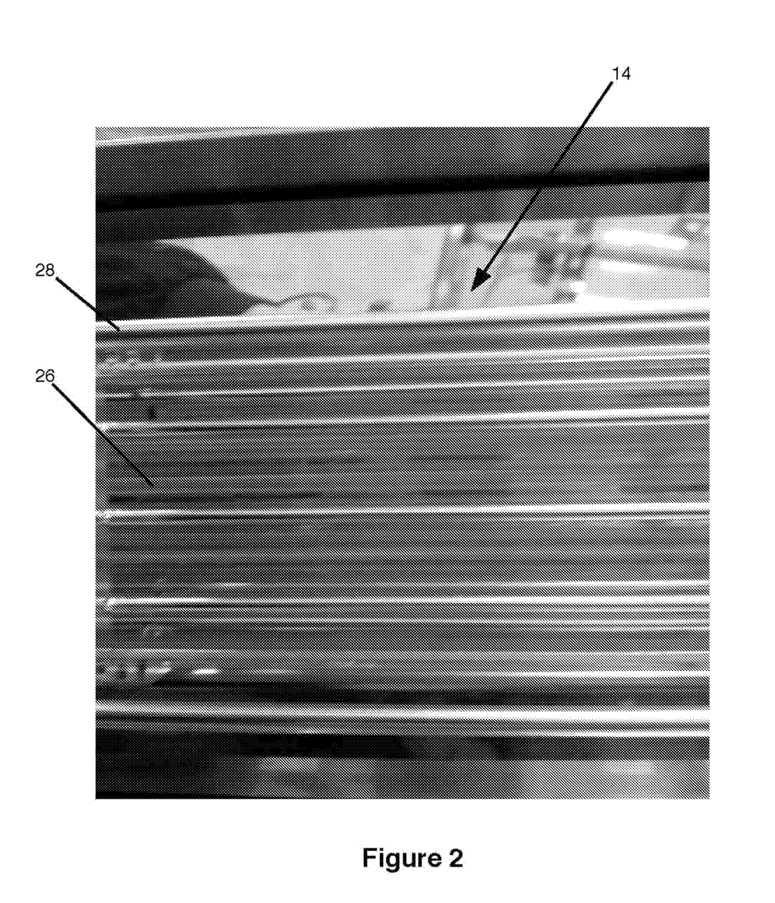

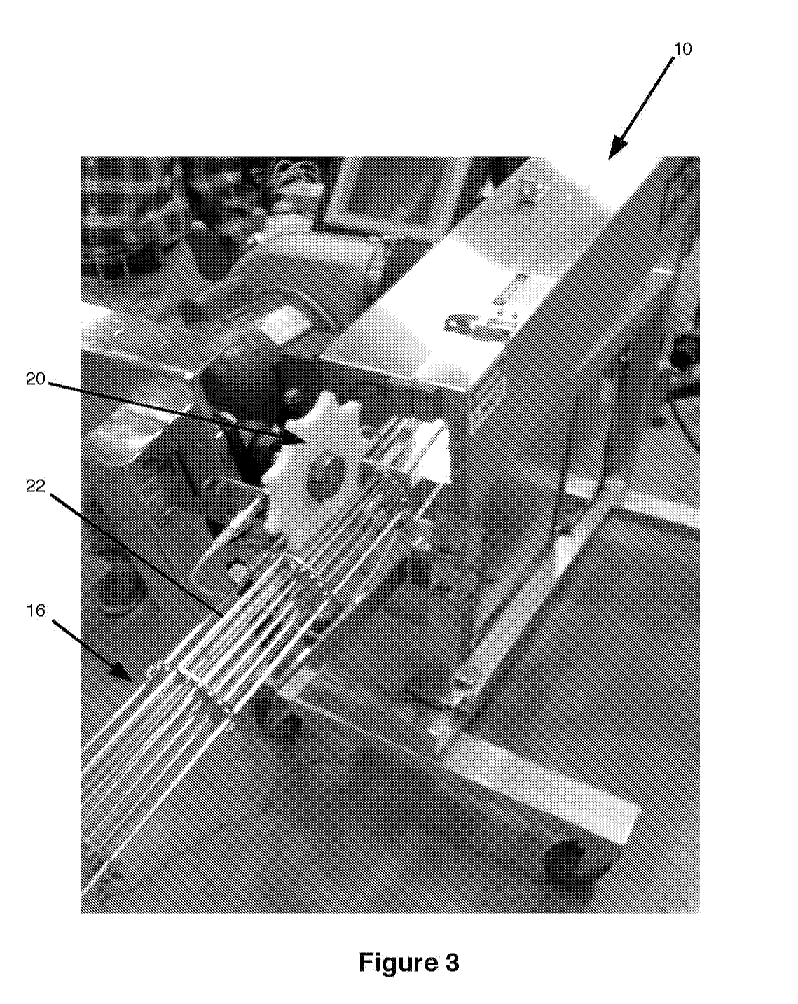

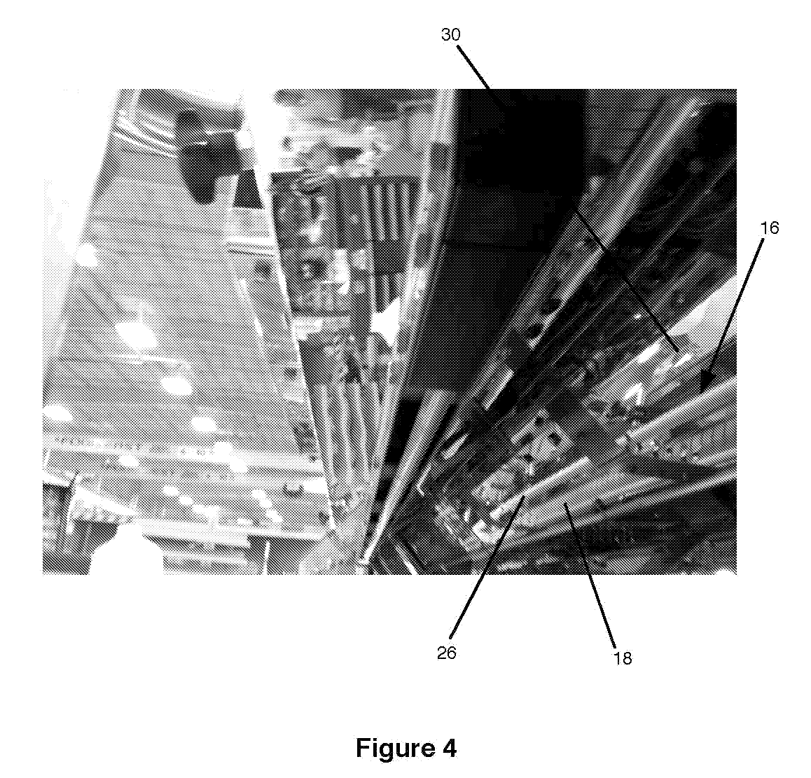

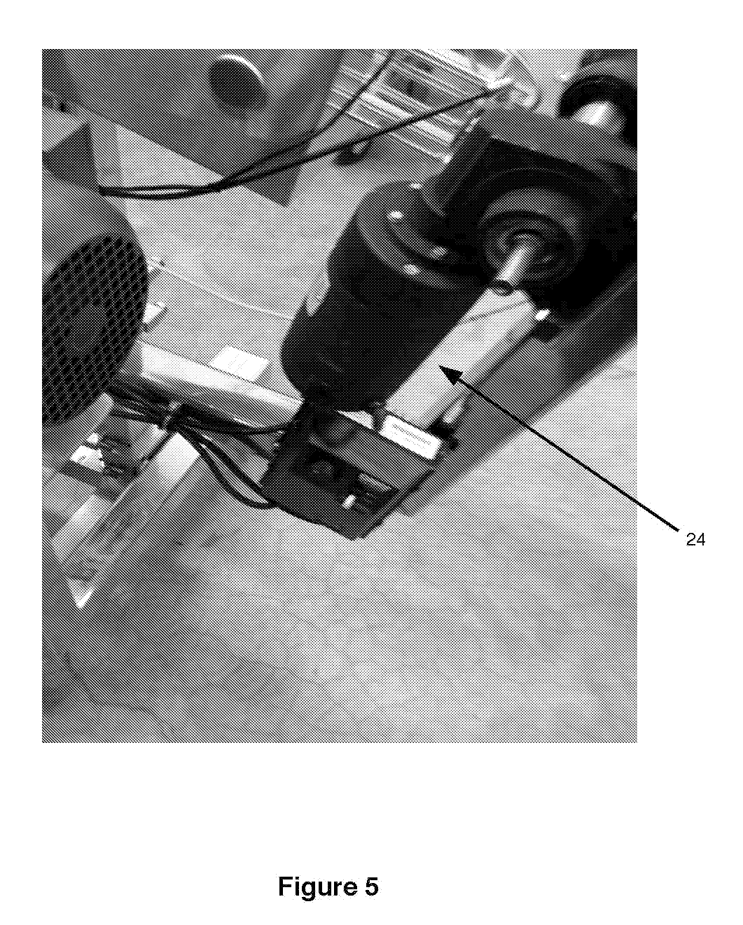

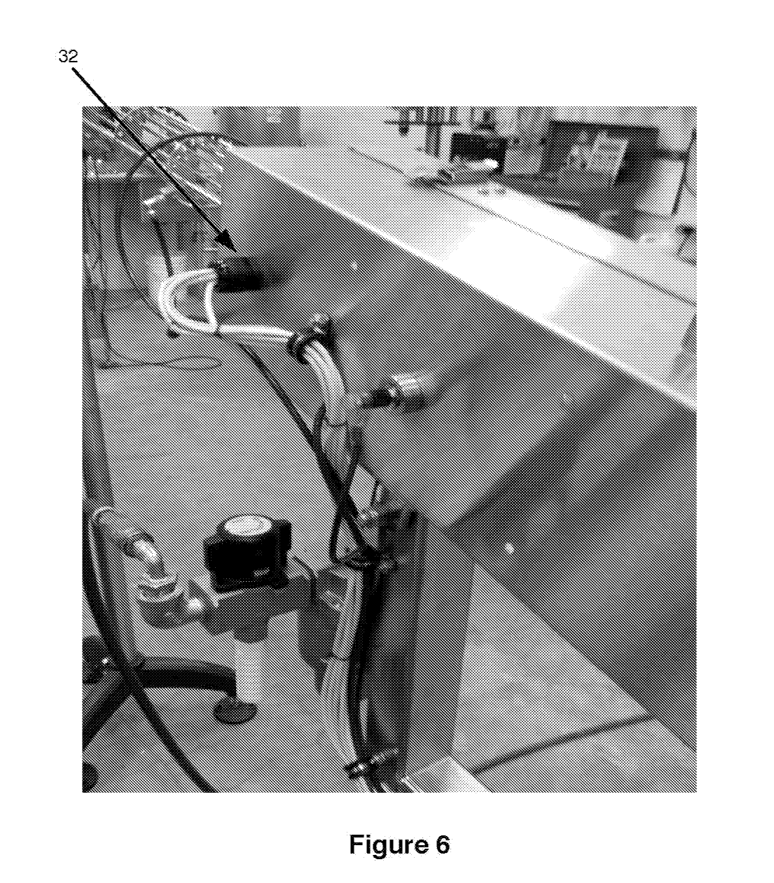

Referring now to the collective drawings (i.e., FIGS. 1-6), and to FIG. 1 in particular, shown therein is a perspective view of an ultraviolet sanitization and sterilization apparatus, hereinafter referred to as apparatus 10. In one embodiment, apparatus 10 sanitizes and/or sterilizes plastic beverage container components such as bottle caps, although one of ordinary skill in the art will readily appreciate that many other types of objects may be sanitized or sterilized by apparatus 10.

In one particular embodiment, apparatus 10 receives beverage container caps from a beverage container cap feeder (not shown), sanitizes and/or sterilizes the beverage container caps, and communicates the sanitized and/or sterilized beverage container caps to bottling apparatus (also not shown) which joins the sanitized and/or sterilized beverage container caps to beverage containers.

It will be understood that apparatus 10 is preferably capable of between 2 and 5+ Log reduction in the amount of “undesirable matter” present on objects communicating through apparatus 10. In particular, operating apparatus 10 at a first temperature and for a predetermined period of time causes between 2 and 4 Log reductions (sanitization) in the amount of undesirable matter on the objects. Additionally, operating apparatus 10 at either: (1) a temperature greater than the first temperature; or (2) for a longer period of time will result in a 5+ Log reduction (sterilization) in the amount of undesirable matter on the objects. It will be further understood that the term “undesirable matter” includes for example, microorganisms, bacteria, fungi, and/or any other neutralizable matter that is deemed unacceptable on or within any part of an object utilized in the food and/or beverage industries, or other industries such as medical device industries, computer component industries, or other similar industries.

In one embodiment, apparatus 10 includes housing 12 that in one embodiment is preferably constructed from one or more materials capable of resisting and/or reducing the adhesion and/or multiplication of undesirable material, such as stainless steel, copper, and brass—just to name a few. Furthermore, the material or materials selected for constructing housing 12 may preferably reflect rather than absorb UV light.

In accordance with the present invention, housing 12 is a hollow rectangular member configured to enclose UV source 14 which is positioned adjacent to track 16 that extends laterally through housing 12. It will be understood that the shape and configuration of housing 12 may vary according to design requirements such as the size and shape of the objects communicating through apparatus 10. In some embodiments, track 16 may include belt type conveyor 18 adapted to communicate objects linearly through housing 12. Track 16 may also include at least one gear or cog 20 adapted to aid in the communication of objects along conveyor 18. Cog 20 may be provided with a plurality of teeth that are spaced apart from one another such that as cog 20 turns each tooth contacts a separate object communicating along conveyor 18, urging the object into track 16 extending from housing 12. Moreover, at least a portion of each tooth extends into track 16 to contact objects communicating therethrough.

At least a portion of track 16 may be preferably constructed from the same material or materials as housing 12 and in one embodiment at least a portion of track 16 includes a plurality of elongated rods 22 arranged in spaced apart relationship to one another to form a path for communication of objects therethrough. Objects communicating through track 16 via conveyor 18 may contact portions of rods 22, therefore the diameter of rods 22 can be minimized to reduce the obstruction of UV light contacting the objects traveling through track 16 and along conveyor 18.

According to some embodiments, portions of apparatus 10 such as housing 12 and track 16 may be selectively adjustable to vary the angle of such objects relative to the ground. As such, apparatus 10 may include hydraulic assembly 24 that is operatively coupled to housing 12.

In one embodiment, UV source 14 includes a plurality of elongated UV bulbs 26 capable of producing at least one of UV-A, B, and C light. Non-limiting suitable examples of UV bulbs 26 for use with apparatus 10 may include any commercially available non-xenon germicidal UV bulbs 26 available from such companies as Osram Sylvania and Phillips Global. UV bulbs 26 are held in spaced apart relationship to one another at a distance away from conveyor 18 via frame 28. In one embodiment, UV bulbs 26 are positioned in a substantially arcuate pattern such that when housing 12 is disposed in a closed configuration, UV bulbs 26 surround at least a portion of conveyor 18 and extend laterally along the length of conveyor 18. It will be understood that frame 28 may be constructed from material similar to housing 12. Furthermore, brackets or clips of frame 28 utilized to retain UV bulbs 26 may be sized to reduce interference with the transmission and reflection of UV light within housing 12.

According to some embodiments, one or more UV bulbs 26 and reflective plate 30 may be disposed on opposing sides of conveyor 18 in addition to UV bulbs 26 attached to frame 28.

In general, UV bulbs 26 have declining temperature profiles such that when initially energized the UV-C light output of UV bulbs 26 reaches a maximum output level and thereafter declines to an output level that is substantially constant, but sometimes lower than maximum output level. This phenomenon may cause deleterious reductions in the sanitizing and/or sterilizing capabilities of UV bulbs 26 as compared to UV bulbs 26 operating at substantially maximum output.

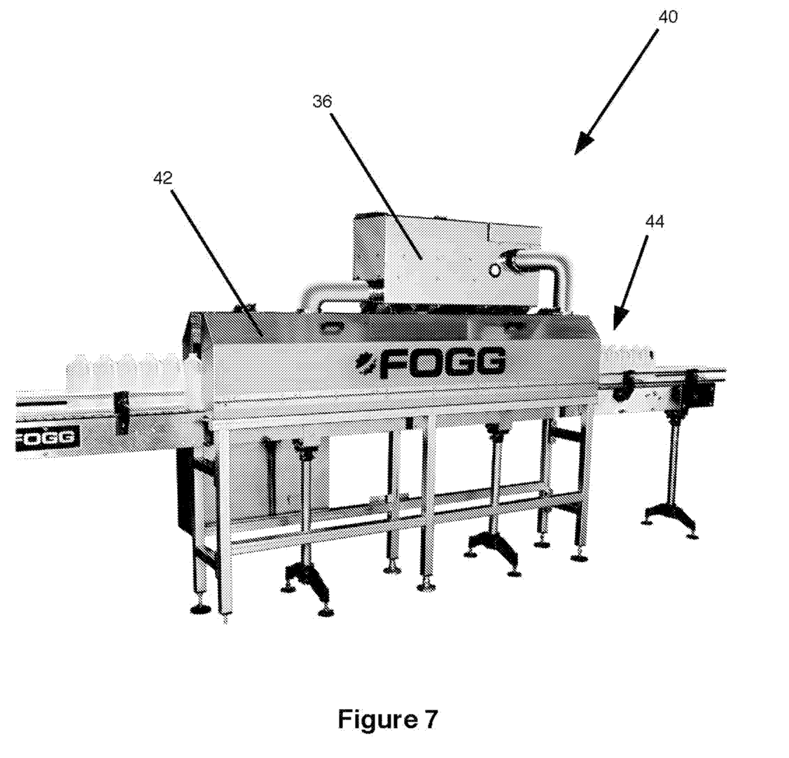

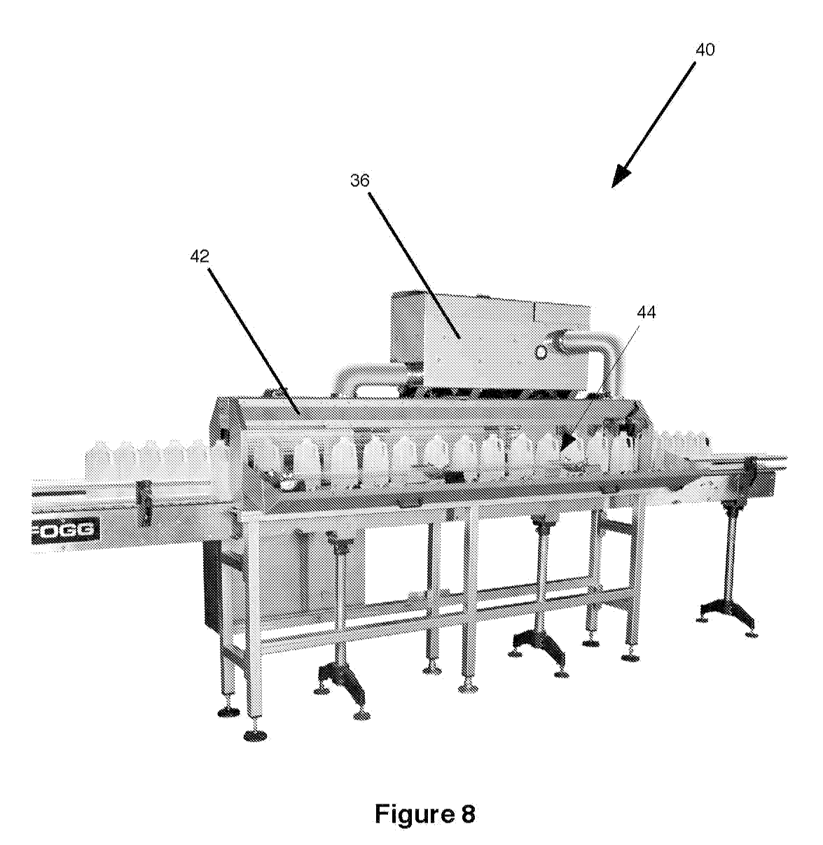

Therefore, to maintain UV bulbs 26 at maximum output levels and cause efficient reductions of undesirable matter, apparatus 10 includes air mover 32, sensor 34, and HEPA filtering system 36 (best shown in FIGS. 7 and 8).

According to some embodiments, air mover 32 may be any number of devices capable of delivering a continuous or intermittent flow of air into housing 12 such as a fan, blower, or the like. In additional embodiments, sensor 34 may include a photospectrometer capable of sensing UV light, and particularly the magnitude of UV-C light emitted by UV bulbs 26. It will be understood that apparatus 10 may include one or more sensors 34 capable of sensing other types of electromagnetic radiation, and according to some embodiments, sensor 34 may be adapted to detect the amount of UV-C light output by UV bulbs 26. In additional embodiments, sensor 34 may include additional types of sensors for detecting properties such as fluid flow rates for measuring the airflow within housing 12.

It will be understood that apparatus 10 operates more efficiently when the air within housing 12 is substantially free from particulate foreign matter. Therefore, in another embodiment, apparatus 10 includes an optional HEPA filtering system 36 which may include a HEPA filter for purifying the air circulating within housing 12. HEPA filtering system 36 may be installed in series with the air mover 32 to filter air introduced into housing 12 or can be installed separately from air mover 32 to create a closed loop of filtered air within apparatus 10.

In operation, objects such as beverage bottle caps may be introduced onto conveyor 18 and communicated through housing 12, during communication of the objects through housing 12, air mover 32, sensor 34 and HEPA filtering system 36 cooperate to sustain UV bulbs 26 at sustained and maximum output levels such that apparatus 10 may cause between 2 and 5+ Log reduction in the amount of “undesirable matter” present on objects communicating through apparatus 10. The sanitized and/or sterilized objects are then urged by cog 20 into track 16 extending beyond housing 12 towards a beverage container capping apparatus (not shown) where the objects are associated with a beverage container.

FIGS. 7 and 8 illustrate a UV sanitization and sterilization apparatus 40 that functions similarly to apparatus 10 with the exception that housing 42 and track 44 are adapted to receive and communicate beverage containers. As such, track 44 may not include elongated rods extending from housing 42.

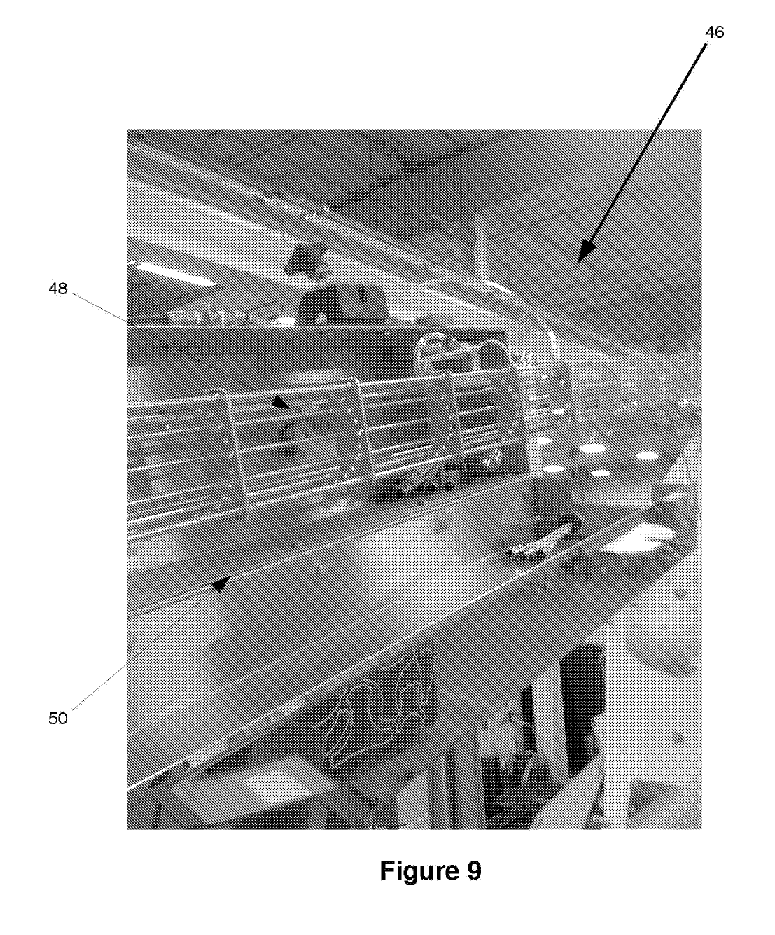

Referring now to FIG. 9, in accordance with the present invention, gravity fed UV sanitization and sterilization apparatus 46 is shown. Apparatus 46 may be constructed similarly to apparatus 10 with the exception that track 48 extends laterally through housing 50 and does not include a conveyor. Rather, housing 50 and track 48 may be disposed at a predetermined angle relative to the ground to allow objects to be gravity fed through housing 50 along track 48. It will be understood that the shape and configuration of housing 50 may vary according to design requirements such as the size and shape of the objects communicating through apparatus 46.

The foregoing description merely explains and illustrates the invention and the invention is not limited thereto except insofar as the appended claims are so limited, as those skilled in the art who have the disclosure before them will be able to make modifications without departing from the scope of the invention.

What is claimed and desired to be secured by Letters Patent of the United States is:

1. An ultraviolet sanitation and sterilization apparatus as provided in FIGS. 1-9 having one or more of the disclosed structural, functional, and/or ornamental characteristics.

2. A method for operating one or more ultraviolet bulbs at an actual maximum output of the same utilizing an ultraviolet sanitation and sterilization apparatus as provided herein having one or more of the disclosed structural, functional, and/or ornamental characteristics.

3. A method for at least one of sanitizing and sterilizing an object utilizing an ultraviolet sanitation and sterilization apparatus as disclosed herein.
